(19) United States
(12) Patent Application Publication
(10) Pub. No.: US 2026/0217752 A1
(43) Pub. Date: Jul. 30, 2026

(54) 2-HYDROXY AND 4-HYDROXY GLYCOLIPIDS AND THEIR DERIVATIVES

(71) Applicant: GlycoSurf, Inc., Salt Lake City, UT (US)

(72) Inventors: Chett Boxley, Park City, UT (US); Ryan Stolley, Salt Lake City, UT (US); Robert Bruggeman, Salt Lake City, UT (US)

(21) Appl. No.: 19/140,168

(22) PCT Filed: Dec. 19, 2023
(86) PCT No.: PCT/US2023/084948

§ 371 Date: Jun. 17, 2025

Related U.S. Application Data
(60) Provisional application No. 63/433,668, filed on Dec. 19, 2022.
(60) Provisional application No. 63/586,744, filed on Sep. 29, 2023.

(51) Int. Cl. C07H 15/04

(52) U.S. Cl. CPC C07H 15/04

(57) ABSTRACT

Described are glycolipids having a saccharide moiety attached at the 2-position of the lipid chain (i.e., “2-hydroxy glycolipids”), glycolipids having a saccharide moiety attached at the 4-position of the lipid chain (i.e., “4-hydroxy glycolipids”), and derivatives thereof. Further described are synthetic methods for preparing 2-hydroxy glycolipids, 4-hydroxy glycolipids, and derivatives thereof.

### CROSS-REFERENCE TO RELATED APPLICATION

This application claims the benefit of and priority to U.S. Provisional Application No. 63/433,668, filed on Dec. 19, 2022, and claims priority to U.S. Provisional Application No. 63/586,744, filed on Sep. 29, 2023, the entire contents both of which are incorporated herein by reference.

### TECHNICAL FIELD

The present disclosure relates to glycolipids having a saccharide moiety attached at the 2-position of the lipid chain (i.e., “2-hydroxy glycolipids”), glycolipids having a saccharide moiety attached at the 4-position of the lipid chain (i.e., “4-hydroxy glycolipids”), and derivatives thereof. Further disclosed are synthetic methods for preparing 2-hydroxy glycolipids, 4-hydroxy glycolipids, and derivatives thereof.

### INTRODUCTION

Glycolipids are lipids with a saccharide (i.e., sugar or carbohydrate) attached by a glycosidic (covalent) bond. Glycolipids may be naturally occurring or synthetically prepared. However, all naturally occurring glycolipids have the saccharide moiety attached at the 3-position of the lipid chain (i.e., “3-hydroxy glycolipids,”), as illustrated below,

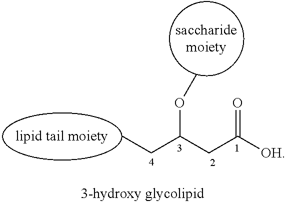

Moreover, to date, glycolipid syntheses and research efforts have centered around these 3-hydroxy glycolipids. What is needed are structurally unique glycolipids and methods of synthetically accessing the same.

### SUMMARY

In some aspects, the present disclosure provides glycolipids of formula (I), or salt thereof,

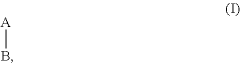

wherein:
   A is a monosaccharide, a disaccharide, a trisaccharide, or an amine or thiol derivative thereof;
   B is

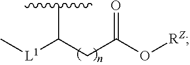

L¹ is C₂-alkylene or C₂₋₁₈alkenylene;
n is 0 or 2;
R^{Z} is hydrogen, C₁₋₆alkyl, C₁₋₆haloalkyl, G^{1a}, —C₁₋₆alkylene-G^{1a}, —C₁₋₆alkylene-R^{Y}, or

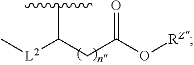

L², at each occurrence, is C₂₋₁₈alkylene or C₂₋₁₈alkenylene;
n″, at each occurrence, is 0 or 2;
R^{Z″}, at each occurrence, is hydrogen, C₁₋₆alkyl, C₁₋₆haloalkyl, G^{1a″}, —C₁₋₆alkylene-G^{1a″}, or —C₁₋₆alkylene-R^{Y″};
R^{Y}, at each occurrence, is —OC₁₋₄alkyl, —OC₁₋₄haloalkyl, —OH, cyano, —SH, —SC₁₋₄alkyl, —SC₁₋₄haloalkyl, —SG^{1a}, —NH₂, —NHC₁₋₄alkyl, —NHG^{1a}, —N(C₁₋₄alkyl)₂, —N(G^{1a})₂, —C(O)OG^{1a}, —C(O)OC₁₋₄alkyl, —C(O)NH₂, —C(O)NHC₁₋₄alkyl, —C(O)N(C₁₋₄alkyl)₂, —C(O)NHG^{1a}, —C(O)N(G^{1a})₂, —SO₂C₁₋₄alkyl, —SO₂G^{1a}, —SO₂NH₂, —SO₂NHC₁₋₄alkyl, or —SO₂N(C₁₋₄alkyl)₂;
R^{Y″}, at each occurrence, is —OC₁₋₄alkyl, —OC₁₋₄haloalkyl, —OH, cyano, —SH, —SC₁₋₄alkyl, —SC₁₋₄haloalkyl, —SG^{1a″}, —NH₂, —NHC₁₋₄alkyl, —NHG^{1a″}, —N(C₁₋₄alkyl)₂, —N(G^{1a″})₂, —C(O)OG^{1a″}, —C(O)OC₁₋₄alkyl, —C(O)NH₂, —C(O)NHC₁₋₄alkyl, —C(O)N(C₁₋₄alkyl)₂, —C(O)NHG^{1a″}, —C(O)N(G^{1a″})₂, —SO₂C₁₋₄alkyl, —SO₂G^{1a″}, —SO₂NH₂, —SO₂NHC₁₋₄alkyl, or —SO₂N(C₁₋₄alkyl)₂; and
G^{1a} and G^{1a″}, at each occurrence, are independently a C₃₋₈cycloalkyl, a 4- to 12-membered heterocyclyl, a 6- to 12-membered aryl, or a 5- to 12-membered heteroaryl, wherein G^{1a} and G^{1a″} are independently optionally substituted with 1-5 substituents independently selected from the group consisting of halogen, oxo, C₁₋₄alkyl, —OC₁₋₄alkyl, —OC₁₋₄haloalkyl, —OH, —SC₁₋₄alkyl, —SC₁₋₄haloalkyl, —SH, —NO₂, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, —C(O)N(C₁₋₄alkyl)₂, —SO₂C₁₋₄alkyl, —SO₂NH₂, —SO₂NHC₁₋₄alkyl, and —SO₂N(C₁₋₄alkyl)₂.

In some instances, the saccharide is a monosaccharide. The monosaccharide may be allose, altrose, arabinose, fructose, fucose, galactose, glucose, gulose, idose, lxyose, psicose, rhamnose, ribose, 2-deoxy-ribose, ribulose, sorbose, tagatose, talose, xylose, xylulose, or an amine or thiol derivative thereof

In some instances, the saccharide is a disaccharide. The disaccharide my be cellobiose, chitobiose, dirhamnose, gentiobiose, isomaltose, isomaltulose, lactose, lactulose, laminaribose, leucrose, maltose, maltulose, melibiose, nigerose, sophorose, sucrose, terhalose, turanose, xylobiose, or an amine or thiol derivative thereof. The saccharide may be a trisaccharide.

In some instances, the saccharide is a trisaccharide. The trisaccharide may be cellotriose, isomaltotriose, isopanose, laminaritriose, manninotriose, maltotriose, melezitose, nigerotriose, panose, raffinose, xylotriose, or an amine or thiol derivative thereof.

In some instances, the saccharide is a naturally occurring saccharide. In other instances, the saccharide is a synthetically prepared saccharide.

In some instances, A is:

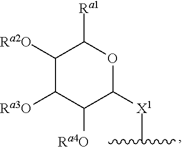

wherein:
R^{a1} is —CH₃, —CH₂OH, or hydrogen;
R^{a2} and R^{a3} are each hydrogen;
R^{a4} is hydrogen, a monosaccharide moiety, or a disaccharide moiety; and
X¹ is O, S, or NH.

In some instances, A is:

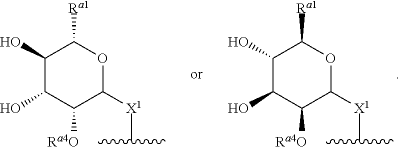

In some instances, A is:

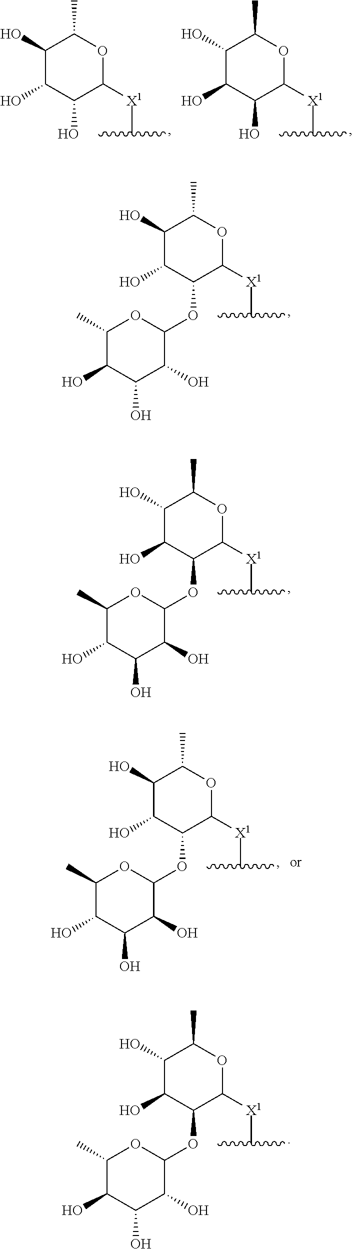

In some instances, X¹ is O.

In some instances, the glycolipid of formula (I) is a glycolipid of formula (I-a), (I-b), (I-aa), (I-bb), (I-ab), or (I-ba):

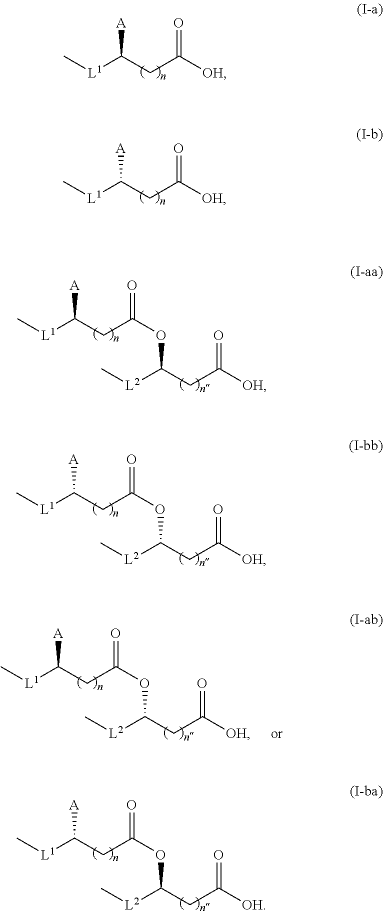

In other aspects, the present disclosure provides methods of preparing glycolipids, or the salt thereof, wherein n is 0, the method comprising:
i. preparing an intermediate of formula (I-1B), where X¹ is O, S, or NH:

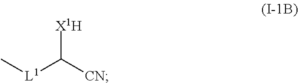

ii. hydrolyzing the intermediate of formula (I-1B) to provide an intermediate of formula (I-1C):

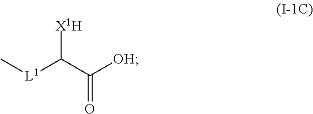

iii. reacting the intermediate of formula (I-1C) with a reagent containing a first hydroxyl protecting group (PG) to provide an intermediate of formula (I-1D):

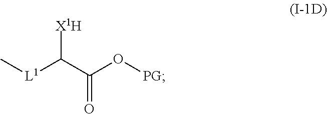

i. in the presence of a glycosylation promoter, reacting the intermediate of formula (I-1D) with a compound of formula (I-S):

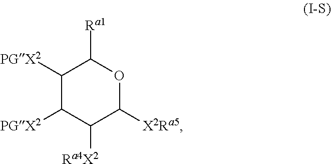

wherein:
   X², at each occurrence, is O or S;
   PG″, at each occurrence is a second hydroxyl protecting group or a thiol protecting group;
   R^{a1} is —CH₃, hydrogen, or —CH₂X²PG″; and
R^{a4} is PG″, a monosaccharide moiety, or a disaccharide moiety,
to provide an intermediate of formula (I-1E):

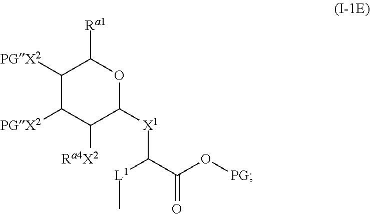

and
iv. removing the PG and PG″ groups from the intermediate of formula (I-2E) to provide a glycolipid of formula:

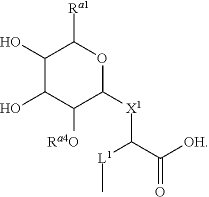

Preparing the intermediate of formula (I-1B) may comprise reacting a nitrile with an intermediate of formula (I-1A):

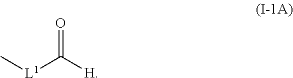

In other aspects, the present disclosure provides methods of preparing glycolipids, or the salt thereof, wherein n is 2, the method comprising:
i. preparing an intermediate of formula (I-2B):

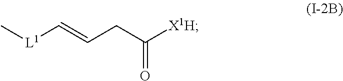

ii. cyclizing the intermediate of formula (I-2B) to provide an intermediate of formula (I-2C:

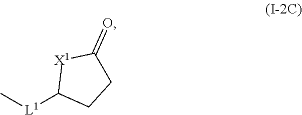

where X¹ is O, S, or NH;
iii. hydrolyzing the intermediate of formula (I-2C) to provide an intermediate of formula (I-2D):

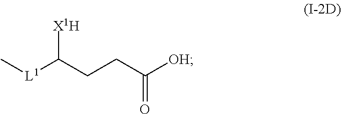

iv. reacting the intermediate of formula (I-2D) with a reagent containing a first hydroxyl protecting group (PG) to provide an intermediate of formula (I-2E):

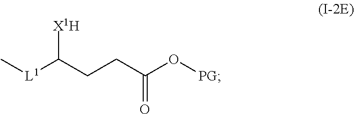

v. in the presence of a glycosylation promoter, reacting the intermediate of formula (I-2E) with a compound of formula (I-S):

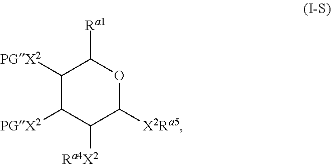

wherein:
   X², at each occurrence is O or S;
   PG″, at each occurrence, is a second hydroxyl protecting group or a thiol protecting group;
   R^{a1} is —CH₃, hydrogen, or —CH₂X²PG″; and
   R^{a4} is PG″, a monosaccharide moiety, or a disaccharide moiety,
to provide an intermediate of formula (I-2F):

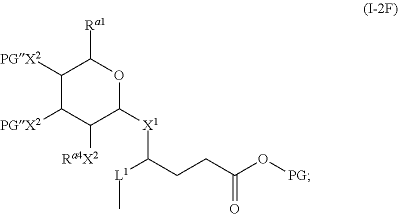

and
vi. removing the PG and PG″ groups from the intermediate of formula (I-2F) to provide a glycolipid of formula:

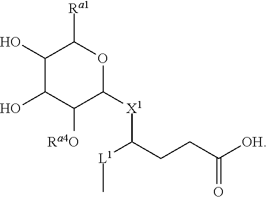

Preparing the intermediate of formula (I-2B) comprises reacting a compound of formula (I-2A′) with a compound of formula (I-2A″):

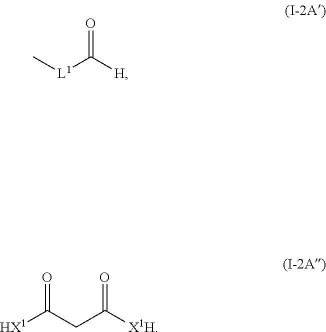

In some instances, the glycosylation promoter comprises Bi(OTf)₃, InBr₃, or BF₃.

In some instances, L¹ may be C₂₋₁₈alkylene.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various way.

### I. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein. The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

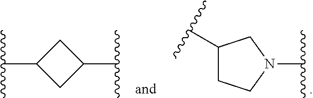 [SMILES: *C1CC(*)C1 ; *C1CCN(*)C1]

Cycloalkylene and heterocyclylene include a geminal divalent groups such as 1,1-C₃₋₆cycloalkylene

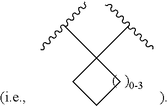

A further example is 1,1-cyclopropylene

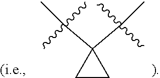 [SMILES: *C1(*)CC1]

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10n electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “nitrile,” as used herein, means any organic compound that has a —C≡N functional group.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

The terms “saccharide,” “sugar,” and “carbohydrate” are used interchangeably herein and generally refer to a mono-, di-, and/or trisaccharide or mixtures thereof.

The term “monosaccharide” refers to any type of hexose of the formula C₆H₁₂O₆ or a derivative thereof. The ring structure (i.e., ring type) of the monosaccharide can be a pyranose or a furanose. In addition, the monosaccharides can be an α- or β-anomer. Monosaccharide can be a ketonic monosaccharide (i.e., ketose), an aldehyde monosaccharide (i.e., aldose), or any type of hexose of the formula C₆H₁₂O₆ or a derivative thereof. Non-limiting examples of monosaccharides of the invention include, but are not limited to, allose, altrose, arabinose, fructose, fucose, galactose, glucose, gulose, idose, lyxose, psicose, rhanmose, ribose, ribulose, sorbose, tagatose, talose, xylose, and xylulose. A monosaccharide may also be an amine or thiol derivative of any monosaccharide. Each monosaccharide may also be independently an (L)-isomer or a (D)-isomer.

The term “disaccharide” refers to a carbohydrate composed of two monosaccharides. It is formed when two monosaccharides are covalently linked to form a dimer. The linkage can be α (1→4) bond, α (1→6) bond, α (1→2) bond, α (1→3) bond, etc. between the two monosaccharides. In addition, each of the monosaccharides can be independently an α- or β-anomer. Non-limiting examples of disaccharides include, but are not limited to, cellobiose, chitobiose, dirhamnose, gentiobiose, isomaltose, isomaltulose, lactose, lactulose, laminaribose, leucrose, maltose, maltulose, melibiose, nigerose, sophorose, sucrose, trehalose, turanose, and xylobiose. A disaccharide may also be an amine or thiol derivative of any monosaccharide units that are linked to make a disaccharide. Each of the monosaccharides can independently be a ketonic monosaccharide (i.e., ketose), an aldehyde monosaccharide (i.e., aldose), or any type of hexose of the formula C₆H₁₂O₆ or a derivative thereof. Each monosaccharide in a disaccharide may also be independently an (L)-isomer or a (D)-isomer.

The term “trisaccharide” refers to a carbohydrate composed of three monosaccharides. It is formed when three monosaccharides are covalently linked to form a trimer. Each of the linkage between monosaccharides can be independently α (1→4) bond, α (1→6) bond, α (1→2) bond, α (1→3) bond, etc. In addition, each of the monosaccharides can be independently an α- or β-anomer. Non-limiting examples of trisaccharides include, but are not limited to, cellotriose, isomaltotriose, isopanose, laminaritriose, manninotriose, maltotriose, melezitose, nige-rotriose, panose, raflinose, and xylotriose. A trisaccharide may also be an amine or thiol derivative of any monosaccharide that are linked to make a trisaccharide. Each of the monosaccharides can independently be a ketonic monosaccharide (i.e., ketose), an aldehyde monosaccharide (i.e., aldose), or any type of hexose of the formula C₆H₁₂O₆ or a derivative thereof. Each monosaccharide within a trisaccharide can also be independently an (L)-isomer or a (D)-isomer.

The term “saccharide derivative,” “sugar derivative,” or “carbohydrate derivative” refers to any chemical modification of the carbohydrate/sugar. Carbohydrate derivatives includes alkylated carbohydrate, replacement of one or more hydroxyl groups with hydrogen, halide, amine, or a thiol; modification of a hydroxyl group (e.g., by esterification, etherification, protection, etc.); as well as other derivatives known to one skilled in the art. The term carbohydrate includes pyranose and furanose carbohydrates. Non-limiting examples of carbohydrate derivatives carbohydrates include, but are not limited to, alkylated carbohydrate (e.g., one or more hydroxyl groups that are methylated, ethylated, acetylated, or benzoylated), thiol carbohydrate (where one or more hydroxyl groups are 10 replaced with —SH moiety), and deoxy carbohydrates (where one or more —OH groups of the carbohydrate is replaced with —H).

The term “a thiol derivative” of a sugar refers to a sugar moiety in which the hydroxyl group that links the sugar to the lipid moiety is replaced with a sulfur atom. Similarly, the term “an amine or amino derivative” of a sugar refers to a sugar moiety in which the hydroxyl group that links the sugar moiety to the lipid moiety is replaced with a nitrogen atom.

### II. Glycolipids

Glycolipids of the present disclosure are set forth in the following numbered embodiments. The first embodiment is denoted E1, another embodiment is denoted E2 and so forth.
E1. A glycolipid of formula (I), or a salt thereof,

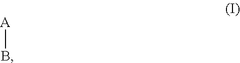

wherein:
A is a monosaccharide, a disaccharide, a trisaccharide, or an amine or thiol derivative thereof;
B is

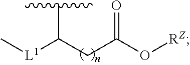

L¹ is C₂-alkylene or C₂₋₁₈alkenylene;
   n is 0 or 2;
R^{Z} is hydrogen, C₁₋₆alkyl, C₁₋₆haloalkyl, G^{1a}, —C₁₋₆alkylene-G^{1a}, —C₁₋₆alkylene-R^{Y}, or

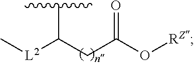

L², at each occurrence, is C₂₋₁₈alkylene or C₂₋₁₈alkenylene;
   n″, at each occurrence, is 0 or 2;
   R^{Z″}, at each occurrence, is hydrogen, C₁₋₆alkyl, C₁₋₆haloalkyl, G^{1″}, —C₁₋₆alkylene-G^{1a″}, or —C₁₋₆alkylene-R^{Y″};
R^{Y}, at each occurrence, is —OC₁₋₄alkyl, —OC₁₋₄haloalkyl, —OH, cyano, —SH, —SC₁₋₄alkyl, —SC₁₋₄haloalkyl, —SG¹, —NH₂, —NHC₁₋₄alkyl, —NHG^{1a}, —N(C₁₋₄alkyl)₂, —N(G^{1a})₂, —C(O)OG^{1a}, —C(O)OC₁₋₄alkyl, —C(O)NH₂, —C(O)NHC₁₋₄alkyl, —C(O)N(C₁₋₄alkyl)₂, —C(O)NHG^{1a}, —C(O)N(G^{1a})₂, —SO₂C₁₋₄alkyl, —SO₂G^{1a}, —SO₂NH₂, —SO₂NHC₁₋₄alkyl, or —SO₂N(C₁₋₄alkyl)₂;
R^{Y″}, at each occurrence, is —OC₁₋₄alkyl, —OC₁₋₄haloalkyl, —OH, cyano, —SH, —SC₁₋₄alkyl, —SC₁₋₄haloalkyl, —SG^{1a″}, —NH₂, —NHC₁₋₄alkyl, —NHG^{1a″}, —N(C₁₋₄alkyl)₂, —N(G^{1a″})₂, —C(O)OG^{1a″}, —C(O)OC₁₋₄alkyl, —C(O)NH₂, —C(O)NHC₁₋₄alkyl, —C(O)N(C₁₋₄alkyl)₂, —C(O)NHG^{1a″}, —C(O)N(G^{1a″})₂, —SO₂C₁₋₄alkyl, —SO₂G^{1a″}, —SO₂NH₂, —SO₂NHC₁₋₄alkyl, or —SO₂N(C₁₋₄alkyl)₂; and G^{1a} and G^{1a″}, at each occurrence, are independently a C₃₋₈cycloalkyl, a 4- to 12-membered heterocyclyl, a 6- to 12-membered aryl, or a 5- to 12-membered heteroaryl, wherein G^{1a} and G^{1a″} are independently optionally substituted with 1-5 substituents independently selected from the group consisting of halogen, oxo, C₁₋₄alkyl, —OC₁₋₄alkyl, —OC₁₋₄haloalkyl, —OH, —SC₁₋₄alkyl, —SC₁₋₄haloalkyl, —SH, —NO₂, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, —C(O)N(C₁₋₄alkyl)₂, —SO₂C₁₋₄alkyl, —SO₂NH₂, —SO₂NHC₁₋₄alkyl, and —SO₂N(C₁₋₄alkyl)₂.
E2. The glycolipid of E1, or the salt thereof, wherein the saccharide is a monosaccharide.
E3. The glycolipid of E2, or the salt thereof, wherein the monosaccharide is allose, altrose, arabinose, fructose, fucose, galactose, glucose, gulose, idose, lxyose, psicose, rhamnose, ribose, 2-deoxy-ribose, ribulose, sorbose, tagatose, talose, xylose, xylulose, or an amine or thiol derivative thereof.
E4. The glycolipid of E1, or the salt thereof, wherein the saccharide is a disaccharide.
E5. The glycolipid of E4, or the salt thereof, wherein the disaccharide is cellobiose, chitobiose, dirhamnose, gentiobiose, isomaltose, isomaltulose, lactose, lactulose, laminaribose, leucrose, maltose, maltulose, melibiose, nigerose, sophorose, sucrose, terhalose, turanose, xylobiose, or an amine or thiol derivative thereof.
E6. The glycolipid of E1, or the salt thereof, wherein the saccharide is a trisaccharide.
E7. The glycolipid of E6, or the salt thereof, wherein the trisaccharide is cellotriose, isomaltotriose, isopanose, laminaritriose, manninotriose, maltotriose, melezitose, nigerotriose, panose, raffinose, xylotriose, or an amine or thiol derivative thereof.
E8. The glycolipid of any one of E1-E7, or the salt thereof, wherein the saccharide is a naturally occurring saccharide.
E9. The glycolipid of any one of E1-E8, or the salt thereof, wherein the saccharide is a synthetically prepared saccharide.
E10. The glycolipid of any one of E1-E9, or the salt thereof, wherein A is:

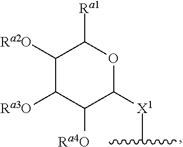

wherein:
R^{a1} is —CH₃, —CH₂OH, or hydrogen;
R^{a2} and R^{a3} are each hydrogen;
R^{a4} is hydrogen, a monosaccharide moiety, or a disaccharide moiety; and
X¹ is O, S, or NH.
E11. The glycolipid of E10, or the salt thereof, wherein A is:

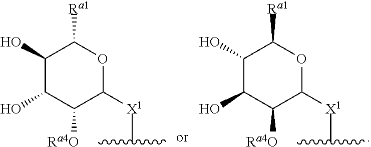

E12. The glycolipid of E11, or the salt thereof, wherein A is:

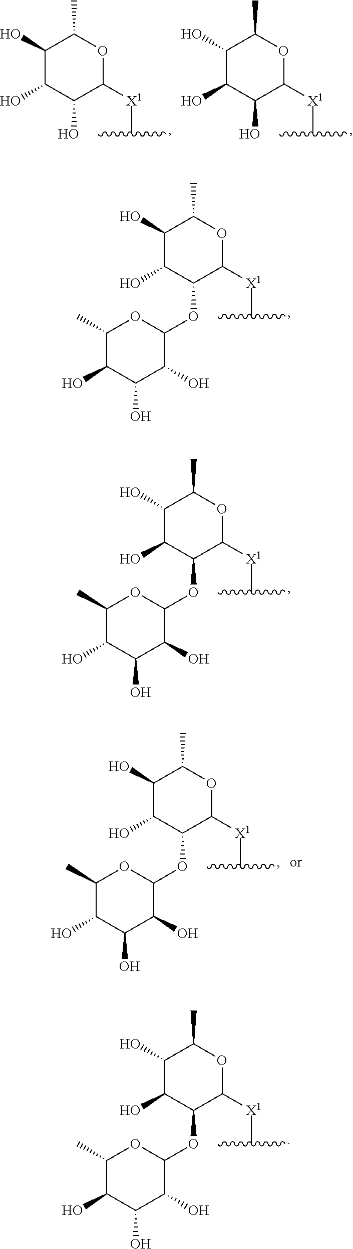

E13. The glycolipid of any one of E1-E12, or the salt thereof, wherein X¹ is O.
E14. The glycolipid of any one of E1-E13, or the salt thereof, wherein the glycolipid of formula (I) is a glycolipid of formula (I-a), (I-b), (I-aa), (I-bb), (I-ab), or (I-ba):

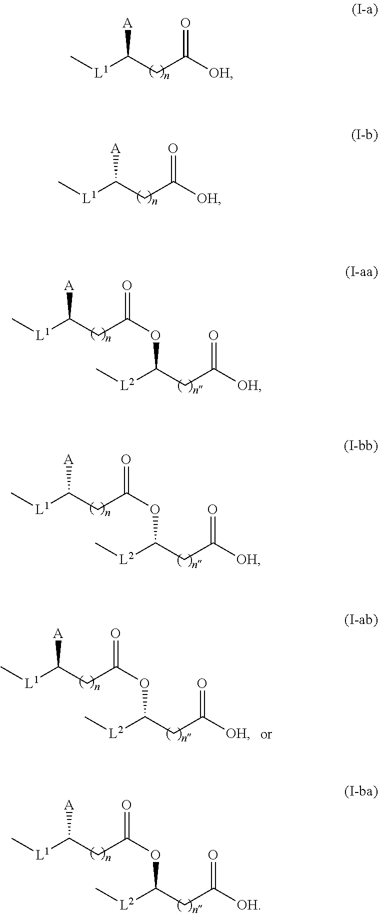

### A. Salts

The disclosed glycolipids may exist as salts, such as pharmaceutically acceptable salts. The term “pharmaceutically acceptable salt” refers to salts or zwitterions of the glycolipids which are water or oil-soluble or dispersible, suitable for administration to a subject (e.g., treatment of disorders) without undue toxicity, irritation, and allergic response, commensurate with a reasonable benefit/risk ratio and effective for their intended use. The salts may be prepared during the final isolation and purification of the glycolipids or separately by reacting an amino group of the glycolipids with a suitable acid. For example, a glycolipid may be dissolved in a suitable solvent and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the glycolipids may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed glycolipids by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

In some instances, the disclosed glycolipids may exist as pharmaceutically acceptable salts. The term “pharmaceutically acceptable salt” refers to salts or zwitterions of the glycolipids which are water or oil-soluble or dispersible, suitable for treatment of disorders without undue toxicity, irritation, and allergic response, commensurate with a reasonable benefit/risk ratio and effective for their intended use.

### B. General Syntheses

In various instances, glycolipids of formula (I) or any of its subformulas may be synthesized as shown in the following schemes.

Abbreviations which have been used in the Schemes that follow are:
TBAI is tetra-n-butylammonium iodide;
TEA is triethylamine;
Pd/C is palladium on carbon;
PG is a protecting group; and
PG″ is a second protecting group.

General Scheme 1, below, illustrates a general method for preparing exemplary glycolipids of formula (I), where n=0.

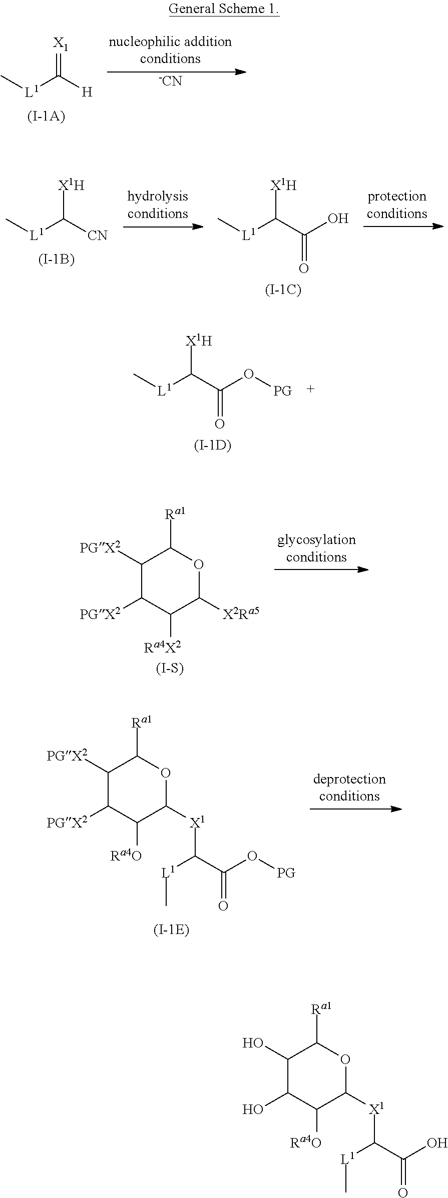

As shown in General Scheme 1 above, in some instances, various glycolipids of formula (I) may be prepared by reacting a compound of formula (I-1A) with a nitrile under suitable nucleophilic addition conditions (e.g., in the presence of a base at a temperature below room temperature) to form an intermediate of formula (I-1B). Intermediates of formula (I-1B) may then be hydrolyzed under suitable hydrolysis conditions (e.g., in the presence of acid heated above room temperature) to form an intermediate of formula (I-1C). Intermediates of formula (I-1C) may then be protected with a suitable hydroxyl protecting group (PG) under suitable protection conditions (e.g., in the presence of TBAI followed by addition of TEA and PG-X, where X is a halogen) to provide intermediates of formula (I-1D). Intermediates of formula (I-1D) may then be reacted with a compound of formula (I-S) under suitable glycosylation conditions (e.g., in the presence of a glycosylation promoter) to produce intermediates of formula (I-1E). Intermediates of formula (I-1E) may be reacted under suitable deprotection conditions (e.g., in the presence of Pd/C and H₂) to provide exemplary glycolipids of formula (I) where n=0.

General Scheme 2, below, illustrates a general method for preparing exemplary glycolipids of formula (I), where n=2.

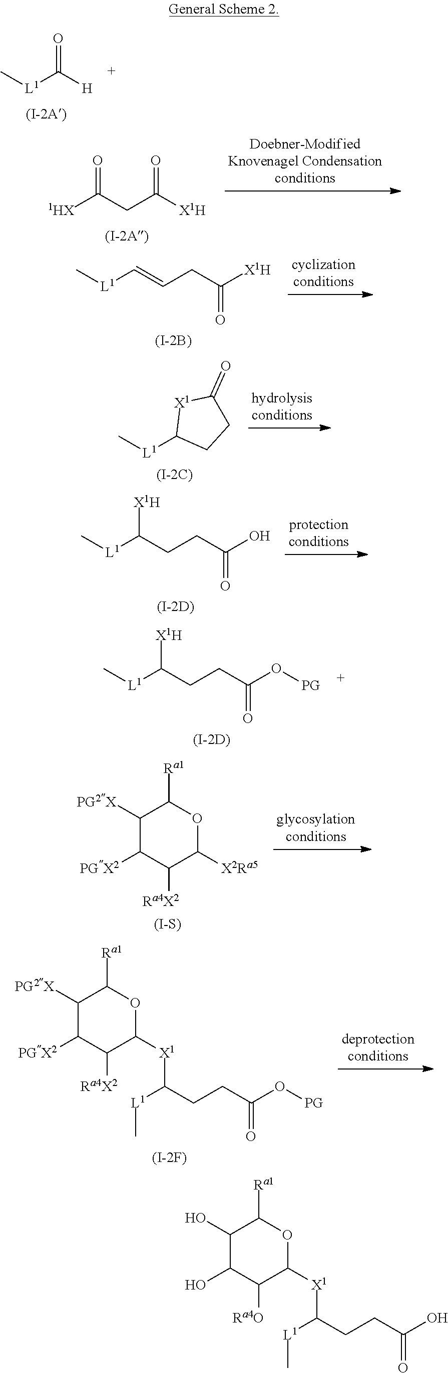

As shown in General Scheme 2 above, glycolipids of formula (I) may be prepared by reacting a compound of formula (I-2A′) with a compound of formula (I-2A″) under suitable condensation conditions, such as Doebner-Modified Knoevenagel condensation conditions (e.g., in the presence of heat and a base) to form an intermediate of formula (I-2B). Intermediates of formula (I-2B) may be cyclized under suitable cyclization conditions (e.g., in the presence of triflic acid and inert gas, heated to reflux) to form an intermediate of formula (I-2C). Intermediates of formula (I-2C) may then be hydrolyzed under suitable hydrolysis conditions (e.g., in the presence of water and a base, such as KOH) to provide intermediates of formula (I-2D). Intermediates of formula (I-2D) may then be protected with a protecting group (PG) under suitable protection condition (e.g., in the presence of TBAI followed by addition of TEA and PG-X, where X is a halogen) to produce intermediates of formula (I-2E). Intermediates of formula (I-2E) may then be reacted with a compound of formula (I-S) under suitable glycosylation conditions (e.g., in the presence of a glycosylation promoter) to form intermediates of formula (I-2F). Intermediates of formula (I-2F) may be reacted under suitable deprotection conditions (e.g., in the presence of Pd/C and H₂) to provide exemplary glycolipids of formula (I) where n=2.

Exemplary glycosylation promoters that may be used in the general syntheses described above are well-known in the art. Exemplary glycosylation promoters include, but are not limited to, bismuth (III) glycolipids (e.g., bismuth (III) trifluoromethanesulfonate, bismuth (III) bromide, bismuth (III) chloride, etc.), scandium(III) glycolipids (e.g., scandium (III) trifluoromethanesulfonate, etc.), boron compounds (e.g., boron trifluoride diethyl etherate), and indium (III) glycolipids (e.g., indium (III) bromide and indium (III) chloride).

The glycolipids and intermediates may be isolated and purified by methods well-known to those skilled in the art of organic synthesis. Examples of conventional methods for isolating and purifying glycolipids can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed glycolipid may have at least one basic nitrogen whereby the glycolipid can be treated with an acid to form a desired salt. For example, a glycolipid may be reacted with an acid at or above room temperature to provide the desired salt, which is deposited, and collected by filtration after cooling. Examples of acids suitable for the reaction may include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above-described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. “Protecting group” refers to a moiety that when attached to a reactive group in a molecule masks, reduces, or prevents that reactivity. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. “Corresponding protecting group” means an appropriate protecting group corresponding to the heteroatom (i.e., N, O, P or S) to which it is attached. Non-limiting examples of hydroxy protecting groups include acyl groups, benzyl and trityl ethers, tetrahydropyranyl ethers, trialkylsilyl ethers and allyl ethers. Non-limiting amino protecting groups include, formyl, acetyl, trifluoroacetyl, benzyl, benzyloxycarbonyl (CBZ), tert-butoxycarbonyl (Boe), trimethyl silyl (TMS), 2-trimethylsilyl-ethanesulfonyl (SES), trityl and substituted trityl groups, allyloxycarbonyl, 9 fluorenylmethyloxycarbonyl (FMOC), nitro-veratryloxycarbonyl (NV OC), and the like. Protecting groups may be included on intermediates, final glycolipids, and reagents used in the chemical syntheses. Synthesis of the glycolipids of the invention can be accomplished by methods analogous to those described in the synthetic schemes described hereinabove and in specific examples.

When an optically active form of a disclosed glycolipid is required, it can be obtained by carrying out one of the procedures described herein using an optically active starting material (prepared, for example, by asymmetric induction of a suitable reaction step), or by resolution of a mixture of the stereoisomers of the glycolipid or intermediates using a standard procedure (such as chromatographic separation, recrystallization, or enzymatic resolution).

Similarly, when a pure geometric isomer of a glycolipid is required, it can be obtained by carrying out one of the above procedures using a pure geometric isomer as a starting material, or by resolution of a mixture of the geometric isomers of the glycolipid or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### C. Applications

Exemplary glycolipids described herein may be used as a surfactant in various products, such as detergents, wetting agents, emulsifiers, foaming agents, and dispersants. Exemplary glycolipids disclosed herein may also be used as a frother and/or a collector (e.g., an ion-collector).

### EXAMPLES

### Example 1: Synthesis of 2-Hydroxy Glycolipids

### Abbreviations

NaHSO₃ is sodium bisfulite;
EtOAc is ethyl acetate;
Et₂O is diethyl ether;
DCM is dichloromethane;
KCN is potassium cyanide;
NaOMe is sodium methoxide;
BF₃-Et₂O is boron trifluoride diethyl ether;
Pd/C is palladium on carbon;
THE is tetrahydrofuran;
NMR is nuclear magnetic resonance;
LRMS is low resolution mass spectrometry;
ESI is electron spray ionization;
TLC is thin-layer chromatography;
eq. or equiv. is equivalents;
min or min. is minute(s);
h or hr. is hour(s);
rt, RT, or r.t. is room temperature; and
sat'd or sat. is saturated.

### A. Synthesis of Example 2-Hydroxy Glycolipid Intermediates

### Example Scheme 1. General Synthesis of Example 2-Hydroxycarboxylic Acids

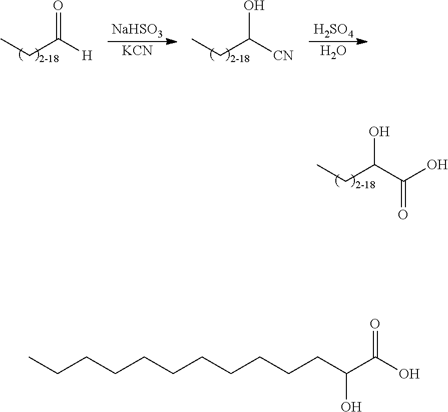

2-Hydroxytridecanoic acid. In a 1-L round bottom flask with large magnetic stir bar dodecanal (25 mL, 0.11 mol, 1 equiv), sodium bisulfite (17.6 g, 0.17 mol, 1.5 equiv) dissolved in 100 mL water, and 200 mL Et₂O were added. The solution was vigorously stirred and cooled to 0° C. Potassium cyanide (9.5 g, 0.1 5 mol, 1.3 equiv) dissolved in 100 mL water was subsequently added dropwise over 30 min. The biphasic solution was vigorously stirred for an additional 2 h. The solution was then added to a separatory funnel and extracted. The aqueous layer was removed, and the remaining organic layer was washed with 3×200 mL water and 100 mL brine. The washed organic layer was dried over sodium sulfate. The solvent was removed by rotary evaporation to afford the intermediate cyanohydrin as a white solid. The solid was then suspended in 250 mL 3 M sulfuric acid and subsequently refluxed for 3 h under nitrogen to produce a dark solution. The dark solution was then added while hot to a separatory funnel, and the aqueous layer acid layer was removed leaving a brown oil. To the brown oil, 200 mL of EtOAc was added. This solution was then washed with 5×100 mL water, 100 mL brine, and dried over sodium sulfate. Solvent was removed under rotary evaporation to afford a light brown solid. This solid was precipitated twice from hexane in a −30° C. freezer, washing each time with cold hexane, to afford the product as a white solid (23 g, 90%—over two steps). ¹H NMR (400 MHz, CDCl₃) δ 12.6 (brs, 1H), 5.62 (brs, 1H), 4.40 (t, 1H), 1.7 (m, 2H), 2.24 (t, 2H), 1.24-1.32 (m, 18H), 0.91 (t, 3H). LRMS (ESI) m/z: [M+H]⁺ Calc'd for C₁₃H₂₅O₃ 229.18; Found 229.22.

### Example Scheme 2. General Synthesis of Example Protected 2-Hydroxycarboxylic Acids

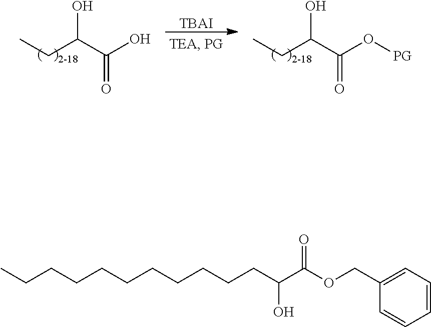

Benzyl 2-hydroxytridecanoate: To a 100-mL round-bottom flask fitted with magnetic stir bar, 2-hydroxytridecanoic acid (4.7 g, 0.021 mol, 1 equiv), TBAI (2 g, 0.003 mol, 0.1 equiv) and 25 mL DMF were added. To this stirred solution, triethylamine (3.6 mL, 0.26 mol, 1.05 equiv) was added and stirred for 10 min at room temperature. Following the addition of triethylamine, benzyl bromide (7.75 mL, 0.03 mol, 1.1 equiv) was added. The solution was heated to 80° C. for 3 h. Upon completion, the reaction was cooled to room temperature and the solution was added to a separatory funnel, along with 200 mL sat'd sodium bicarbonate solution and 100 mL EtOAc. The aqueous fraction was then removed, and the organic fraction was washed with 100 mL sat'd sodium thiosulfate solution, 100 mL water, and 1×100 mL brine. The organic fraction was subsequently dried over sodium sulfate. EtOAc was removed by rotary evaporation to afford an off-white powder. The crude product was then dissolved in minimal amounts of warm hexane and precipitated in a −30° C. freezer overnight. The product was then isolated by vacuum filtration washing with 2×30 mL cold hexane. The product was then air-dried on the frit to afford 6.2 g of benzyl 2-hydroxytridecanoate (95%) as a white powder. ¹H NMR (400 MHz, CDCl₃) δ 7.28-7.33 (m, 5H), 5.58 (brs, 1H), 5.35 (s, 2H), 4.35 (t, 2H), 1.81 (m, 2H), 1.24-1.32 (m, 18H), 0.91 (t, 3H). LRMS (ESI) m/z: [M+H]⁺ Calc'd for C₂₀H₃₃O₃ 321.24; Found 321.18.

### Example Scheme 3. General Synthesis of Example Protected 2-Hydroxycarboxylic Acid Sugars

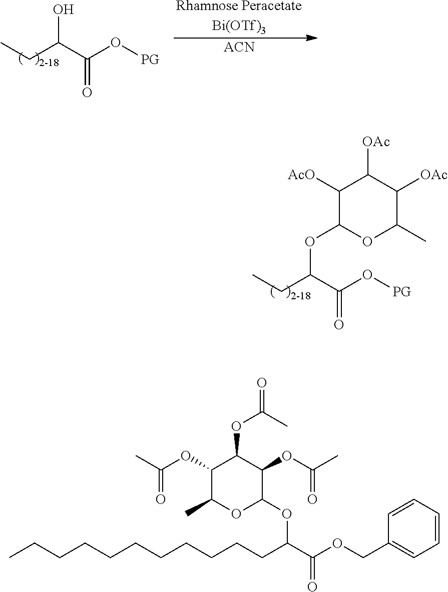

(3R,4R,5S,6S)-2-((1-(Benzyloxy)-1-oxotridecan-2-yl)oxy)-6-methyltetrahydro-2H-pyran-3,4,5-triyl triacetate: To an oven dried 2500-mL round bottom flask equipped with a magnetic stir bar, benzyl 2-hydroxytridecanoate (12 g, 0.037 mol, 1.1 equiv), rhamnose peracetate (11.3 g, 0.034 mol, 1.0 equiv), and 37 mL of dry DCM were added under nitrogen. The resulting solution was cooled to 0° C. and BF₃·Et₂O (4.6 mL, 0.037 mol, 1.1 equiv) was added. The solution was allowed to warm to room temperature overnight. To the stirring solution, 100 mL sat'd sodium bicarbonate solution was added and stirred for 30 min. The resulting biphasic mixture was then added to a separatory funnel and the phases separated. The organic phase was washed with 2×100 mL water, and 1×100 mL brine, and dried over sodium sulfate. The solution was then dried under rotary evaporation to afford the crude product as an amber viscous oil. The crude product purified by column chromatography (10%→50% EtOAc:Hexane) to afford the title diastereomeric mixture as off-white waxy solid (18 g, 82%). LRMS (ESI) m/z: [M+H]⁺ Calc'd for C₃₂H₄₉O₁₀ 593.33; Found 593.41.

### B. Synthesis of Example 2-Hydroxy Glycolipids

### Example Scheme 4. General Synthesis of Example Deprotected 2-Hydroxycarboxylic Acid Sugars

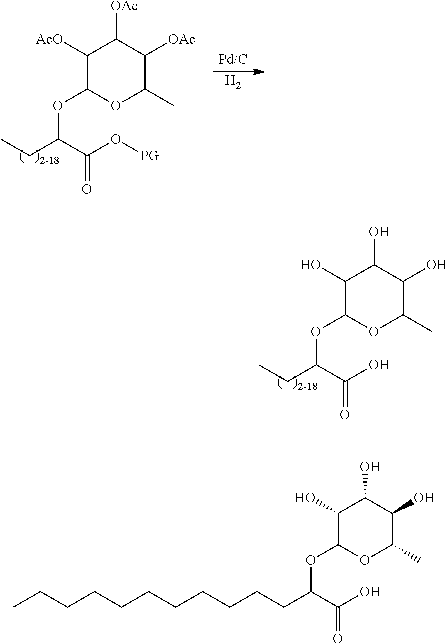

2-(((3R,4R,5R,6S)-3,4,5-Trihydroxy-6-methyltetrahydro-2H-pyran-2-yl)oxy)tridecanoic acid. To a 25-mL scintillation vial equipped with magnetic stir bar, (3R,4R,5S,6S)-2-((1-(benzyloxy)-1-oxotridecan-4-yl)oxy)-6-methyltetrahydro-2H-pyran-3,4,5-triyl triacetate (1.2 g, 2.0 mmol, 1.0 equiv), dry Pd/C (500 mg) and 10 mL THE were added. The vial was fitted with a balloon of hydrogen and the reaction was allowed to proceed for 4 h. Upon completion (TLC), the Pd/C was filtered off and THE removed by rotary evaporation. The resulting debenzylated product was dissolved in dry methanol and NaOMe was added in portions to reach a pH of 11-12. The solution was then stirred at room temperature for 4 hours at which time Dowex-H⁺ was added to a pH of 3-4 at which time the resin was filtered off, and the resulting solution was dried by rotary evaporation. The resulting solid was further dried of solvent for 12 h with a high vacuum to afford 760 mg of a diastereomeric mixture as a white waxy solid (quant.).

LRMS (ESI) m/z: [M+H]⁺ Calc'd for C₁₉H₃₅O₇ 375.24; Found 375.28.

### Example 2: Synthesis of 4-Hydroxy Glycolipids

### Abbreviations

C₇H₁₅NO₂ is piperidinium acetate
DMSO is dimethyl sulfoxide;
EtOAc is ethyl acetate;
TFMS or TfOH is triflic acid;
HCl is hydrochloride acid;
THE is tetrahydrofuran;
TBAI is tetra-n-butylammonium iodide;
TEA is triethylamine;
DMF is dimethylformamide;
NaOMe is sodium methoxide;
Pd/C is palladium on carbon;
Bi(OTf)₃ is bismuth (III) trifluoromethanesulfonate;
Ac is acetate;
NMR is nuclear magnetic resonance;
LRMS is low resolution mass spectrometry;
ESI is electron spray ionization;
TLC is thin-layer chromatography;
PG is protecting group;
eq. or equiv. is equivalents;
min or min. is minute(s);
h or hr. is hour(s);
rt, RT, or r.t. is room temperature; and
sat'd or sat. is saturated.

### A. Synthesis of Example 4-Hydroxy Glycolipid Intermediates

### Example Scheme 5. General Synthesis of Example Carboxylic Acids

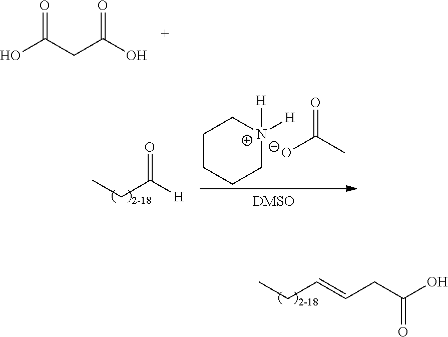

### Example Carboxylic Acid

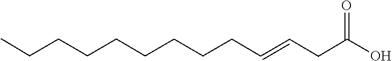

(E)-Tridec-3-enoic acid. To a 250-mL round-bottom flask fitted with magnetic stir bar, malonic acid (28 g, 0.27 mol, 3.0 equiv), dodecanal (20 mL, 0.09 mol, 1.0 equiv), and DMSO (100 mL, ~1M) were added. The reaction mixture was heated to 120° C. and piperidinium acetate (1.3 g, 0.009 mol, 0.1 equiv) was added in a single portion. Within minutes, minor bubbling was observed. The reaction was continued until bubbling stopped (~2 h). The solution was then cooled to rt, added to a separatory funnel and 100 mL 1 M HCl and 400 mL of water was added. The solution was extracted with 3×100 mL EtOAc. The combined organic fractions were then washed with 3×100 mL water and 100 mL brine. The wash organic fractions were dried over sodium sulfate. EtOAc was then removed by rotary evaporation yielding a yellow solid. The crude product was then dissolved in minimal amounts of warm hexane and precipitated in a −30° C. freezer overnight. The product was further dried by vacuum filtration and washing with 2×30 mL cold hexane. The product was then air-dried on the frit to afford 17 g of (E)-dodec-3-enoic acid (95%) as a white powder. 1H NMR (400 MHz, CDCl₃) δ 12.1 (brs, 1H), 5.6-5.8 (m, 2H), 2.86 (d, 2H), 2.14 (dt, 2H), 1.25-1.32 (m, 14H), 0.9 (t, 3H). LRMS (ESI) m/z: [M−H]⁻ Calc'd for C₁₃H₂₂O₂ 211.17; Found 211.20.

### Example Scheme 6. General Synthesis of Example Lactones

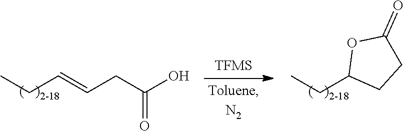

### Example Lactone

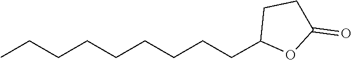

5-Nonyldihydrofuran-2(3H)-one. To a 250-mL round-bottom flask fitted with magnetic stir bar, (E)-dodec-3-enoic acid (24 g, 0.11 mol, 1.0 equiv) and anhydrous toluene (100 mL, ~0.5M) were added under a nitrogen atmosphere. To the stirred solution, triflic acid (TFMS) (9.4 mL, 0.11 mol, 1.0 equiv) at room temperature was added. The solution was then heated to reflux during which the solution becomes dark brown in color. The reaction was refluxed and stirred until complete consumption of the starting material was observed by TLC (1-2 h), The solution was then cooled to room temperature and added to a separatory funnel. The solution was washed with 2×100 mL sat'd sodium bicarbonate solution, 1×100 mL water, and 1×100 mL brine, and dried over sodium sulfate. The solution was then dried under rotary evaporation to afford a brown viscous oil. This oil was precipitated twice from pentane in a −30° C. freezer, washing each time with cold hexane, to afford the product as a white low-melting solid (18 g, 82%). 1H NMR (400 MHz, CDCl₃) δ 4.18 (dt, 1H), 1.95-2.34 (overlapping m, 4H), 1.38 (dt, 2H), 1.25-1.28 (m, 14H), 0.9 (t, 3H). LRMS (ESI) m/z: [M+H]⁺ Calc'd for C₁₂H₂₂O₂ 213.19; Found 213.4.

### Example Scheme 7. General Synthesis of Example 4-Hydroxycarboxylic Acids

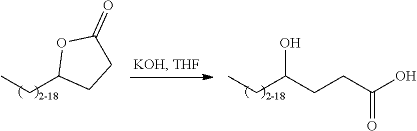

### Example 4-Hydroxycarboxylic Acid

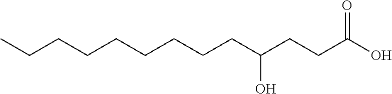 [SMILES: CCCCCCCCCC(O)CCC(=O)O]

4-Hydroxytridecanoic acid. To a 250-mL round-bottom flask fitted with magnetic stir bar, 5-nonyldihydrofuran-2(3H)-one (25 g, 0.118 mol), 50 mL THF, and 50 mL of 3M potassium hydroxide (KOH) were added. The solution was heated to 60° C. for 4 h. Upon completion, the reaction was cooled to room temperature and the solution was added to a separatory funnel. Cold 1 M HCl was added to the separatory funnel until the solution reached a pH of 2. The resulting solution was then extracted with 3×100 mL EtOAc. The combined extracts were subsequently washed with 100 mL water, 2×100 mL sat'd sodium bicarbonate solution, and 1×100 mL brine. The washed organic solution was dried over sodium sulfate. The EtOAc was removed by rotary evaporation to afford an off-white powder. The crude product was then dissolved in minimal amounts of warm hexane and precipitated in a −30° C. freezer overnight. The product was subsequently isolated by vacuum filtration washing with 2×30 mL cold hexane. The product was then air-dried on the frit to afford 17 g of 4-hydroxytridecanoic acid (quant) as a white powder. ¹H NMR (400 MHz, CDCl₃) δ 12.05 (brs, 1H) 4.45 (brs, 1H), 3.39 (m, 1H), 2.32 (t, 2H), 1.72 (dt, 2H), 1.23-1.4 (m, 16H), 0.91 (t, 3H). LRMS (ESI) m/z: [M−H]⁻ Calc'd for C₁₃H₂₅O₃ 229.18; Found 229.25.

### Example Scheme 8. Synthesis of Example Protected 4-Hydroxycarboxylic Acids

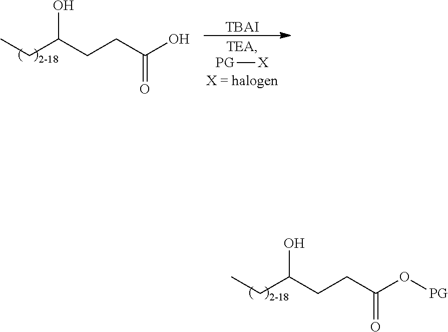

### Example Protected 4-Hydroxycarboxylic Acid

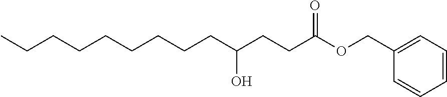 [SMILES: CCCCCCCCCC(O)CCC(=O)OCc1ccccc1]

Benzyl 4-hydroxytridecanoate: To a 100-mL round-bottom flask fitted with magnetic stir bar 4-hydroxytridecanoic acid (6.1 g, 0.024 mol, 1 equiv), TBAI (2 g, 0.003 mol, 0.1 equiv) and 25 mL DMF were added. Triethylamine (3.6 mL, 0.26 mol, 1.05 equiv) was added and stirred for 10 min at room temperature. Following the addition of triethylamine, benzyl bromide (7.75 mL, 0.03 mol, 1.1 equiv) was added to the solution. The solution was heated to 80° C. for 3 h. Upon completion, the reaction was cooled to room temperature and added to a separatory funnel, along with 200 mL sat'd sodium bicarbonate solution and 100 mL EtOAc. The aqueous fraction was removed, and the organic fraction was washed with 100 mL sat'd sodium thiosulfate solution, 100 mL water, 1×100 mL brine, and dried over sodium sulfate. The resulting solution was then dried by rotary evaporation to afford an off-white powder. The crude product was then dissolved in minimal amounts of warm hexane and precipitated in a −30° C. freezer overnight. The product was further isolated by vacuum filtration washing with 2×30 mL cold hexane. The product was then air-dried on the frit to afford 7.2 g of benzyl 4-hydroxytridecanoate (90%) as a white powder. [00100]¹H NMR (400 MHz, CDCl₃) δ 7.18-7.32 (m, 5H), 4.65 (brs, 1H), 4.10 (m, 1H), 3.71 (s, 2H), 2.24 (t, 2H), 1.24-1.42 (m, 18H), 0.88 (t, 3H). LRMS (ESI) m/z: [M+H]⁺ Calc'd for C₂₀H₃₃O₃ 321.24; Found 321.5.

### Example Scheme 9. Synthesis of Example Acetyl-Protected 4-Hydroxycarboxilic Acid Sugars

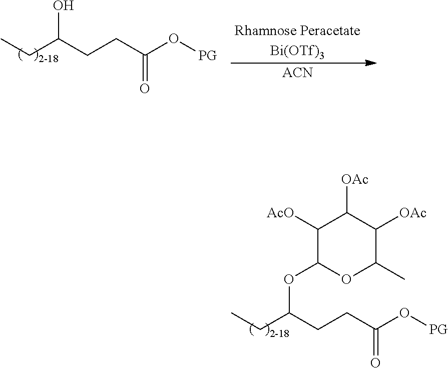

### Example Acetyl-Protected 4-Hydroxycarboxilic Acid Sugar

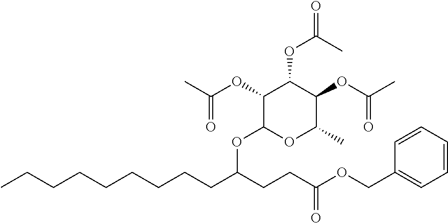 [SMILES: CCCCCCCCCC(CCC(=O)OCc1ccccc1)OC1O[C@@H](C)[C@H](OC(C)=O)[C@@H](OC(C)=O)[C@H]1OC(C)=O]

(3R,4R,5S,6S)-2-((1-(Benzyloxy)-1-oxotridecan-4-yl)oxy)-6-methyltetrahydro-2H-pyran-3,4,5-triyl triacetate. To a 25-mL scintillation vial equipped with magnetic stir bar, benzyl 4-hydroxytridecanoate (750 mg, 2.24 mmol, 1.0 equiv), rhamnose peracetate (964 mg, 2.9 mmol, 1.2 equiv), and 5 mL dry acetonitrile were added. The reaction mixture was then heated to 80° C., and Bi(OTf)₃ (100 mg, 0.1 mmol, 0.05 equiv) was added in a single portion. The reaction proceeded for 30 min and was subsequently cooled to room temperature. To the cooled solution, 8 g of silica were added. The solution was then dried by rotary evaporation. The resultant solids were added to a chromatography column and the mixture was separated by column chromatography (10%→50% EtOAc:Hexane) to afford the title diastereomeric mixture as light-yellow resin (607 mg, 72%). LRMS (ESI) m/z: [M+H]⁺ Calc'd for C₃₂H₄₉O₁₀ 593.33; Found 593.41.

### B. Synthesis of Example 4-Hydroxy Glycolipids

### Scheme 10. General Synthesis of Example Deprotected 4-Hydroxycarboxylic Acid Sugars

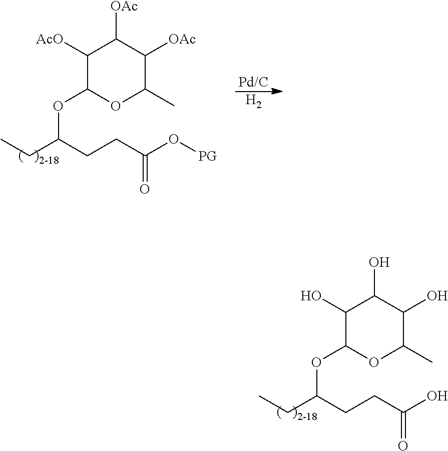

### Example Deprotected 4-Hydroxycarboxylic Acid Sugar

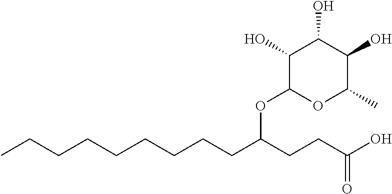 [SMILES: CCCCCCCCCC(CCC(=O)O)OC1O[C@@H](C)[C@H](O)[C@@H](O)[C@H]1O]

4-(((3R,4R,5R,6S)-3,4,5-Trihydroxy-6-methyltetrahydro-2H-pyran-2-yl)oxy)tridecanoic acid. To a 25-mL scintillation vial equipped with magnetic stir bar, (3R,4R,5S,6S)-2-((1-(benzyloxy)-1-oxotridecan-4-yl)oxy)-6-methyltetrahydro-2H-pyran-3,4,5-triyl triacetate (956 mg, 1.6 mmol, 1.0 equiv), dry Pd/C (500 mg) and 5 mL THF were added. The vial was fitted with a balloon of hydrogen and the reaction was allowed to proceed for 4 h. Upon completion (as determined with TLC), the Pd/C was filtered off and THE was removed by rotary evaporation. The resulting debenzylated product was dissolved in dry methanol, and the pH was adjusted to 11-12 with NaOMe. The solution was then stirred at room temperature for 4 hours at which time Dowex-H⁺ was added to a pH of 3-4 at which time the resin was filtered off and the resulting solution was dried by rotary evaporation followed by 12 h on high vacuum to afford 607 mg of a diastereomeric mixture as a white waxy solid (quant.). LRMS (ESI) m/z: [M+H]⁺ Calc'd for C₁₉H₃₅O₇ 375.24; Found 375.33.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:
Clause 1. A glycolipid of formula (I), or a salt thereof,

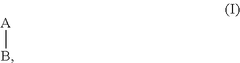

wherein:
   A is a monosaccharide, a disaccharide, a trisaccharide, or an amine or thiol derivative thereof;
   B is

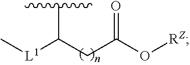

L¹ is C₂-alkylene or C₂₋₁₈alkenylene;
n is 0 or 2;
R^{Z} is hydrogen, C₁₋₆alkyl, C₁₋₆haloalkyl, G^{1a}, —C₁₋₆alkylene-G^{1a}, —C₁₋₆alkylene-R^{Y}, or

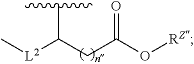

L², at each occurrence, is C₂₋₁₈alkylene or C₂₋₁₈alkenylene;
n″, at each occurrence, is 0 or 2;
R^{Z″}, at each occurrence, is hydrogen, C₁₋₆alkyl, C₁₋₆haloalkyl, G^{1a″}, —C₁₋₆alkylene-G^{1a″}, or —C₁₋₆alkylene-R^{Y″};
R^{Y}, at each occurrence, is —OC₁₋₄alkyl, —OC₁₋₄haloalkyl, —OH, cyano, —SH, —SC₁₋₄alkyl, —SC₁₋₄haloalkyl, —SG^{1a}, —NH₂, —NHC₁₋₄alkyl, —NHG^{1a}, —N(C₁₋₄alkyl)₂, —N(G^{1a})₂, —C(O)OG^{1a}, —C(O)OC₁₋₄alkyl, —C(O)NH₂, —C(O)NHC₁₋₄alkyl, —C(O)N(C₁₋₄alkyl)₂, —C(O)NHG^{1a}, —C(O)N(G^{1a})₂, —SO₂C₁₋₄alkyl, —SO₂G^{1a}, —SO₂NH₂, —SO₂NHC₁₋₄alkyl, or —SO₂N(C₁₋₄alkyl)₂;
R^{Y″}, at each occurrence, is —OC₁₋₄alkyl, —OC₁₋₄haloalkyl, —OH, cyano, —SH, —SC₁₋₄alkyl, —SC₁₋₄haloalkyl, —SG^{1a″}, —NH₂, —NHC₁₋₄alkyl, —NHG^{1a″}, —N(C₁₋₄alkyl)₂, —N(G^{1a″})₂, —C(O)OG^{1a″}, —C(O)OC₁₋₄alkyl, —C(O)NH₂, —C(O)NHC₁₋₄alkyl, —C(O)N(C₁₋₄alkyl)₂, —C(O)NHG^{1a″}, —C(O)N(G^{1a″})₂, —SO₂C₁₋₄alkyl, —SO₂G^{1a″}, —SO₂NH₂, —SO₂NHC₁₋₄alkyl, or —SO₂N(C₁₋₄alkyl)₂; and
G^{1a} and G^{1a″}, at each occurrence, are independently a C₃₋₈cycloalkyl, a 4- to 12-membered heterocyclyl, a 6- to 12-membered aryl, or a 5- to 12-membered heteroaryl, wherein G^{1a} and G^{1a″} are independently optionally substituted with 1-5 substituents independently selected from the group consisting of halogen, oxo, C₁₋₄alkyl, —OC₁₋₄alkyl, —OC₁₋₄haloalkyl, —OH, —SC₁₋₄alkyl, —SC₁₋₄haloalkyl, —SH, —NO₂, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, —C(O)N(C₁₋₄alkyl)₂, —SO₂C₁₋₄alkyl, —SO₂NH₂, —SO₂NHC₁₋₄alkyl, and —SO₂N(C₁₋₄alkyl)₂.
Clause 2. The glycolipid of clause 1, or the salt thereof, wherein the saccharide is a monosaccharide.
Clause 3. The glycolipid of clause 2, or the salt thereof, wherein the monosaccharide is allose, altrose, arabinose, fructose, fucose, galactose, glucose, gulose, idose, lxyose, psicose, rhamnose, ribose, 2-deoxy-ribose, ribulose, sorbose, tagatose, talose, xylose, xylulose, or an amine or thiol derivative thereof.
Clause 4. The glycolipid of clause 1, or the salt thereof, wherein the saccharide is a disaccharide.
Clause 5. The glycolipid of clause 4, or the salt thereof, wherein the disaccharide is cellobiose, chitobiose, dirhamnose, gentiobiose, isomaltose, isomaltulose, lactose, lactulose, laminaribose, leucrose, maltose, maltulose, melibiose, nigerose, sophorose, sucrose, terhalose, turanose, xylobiose, or an amine or thiol derivative thereof.
Clause 6. The glycolipid of clause 1, or the salt thereof, wherein the saccharide is a trisaccharide.
Clause 7. The glycolipid of clause 6, or the salt thereof, wherein the trisaccharide is cellotriose, isomaltotriose, isopanose, laminaritriose, manninotriose, maltotriose, melezitose, nigerotriose, panose, raffinose, xylotriose, or an amine or thiol derivative thereof.
Clause 8. The glycolipid of clauses 1-7, or the salt thereof, wherein the saccharide is a naturally occurring saccharide.
Clause 9. The glycolipid of clauses 1-7, or the salt thereof, wherein the saccharide is a synthetically prepared saccharide.
Clause 10. The glycolipid of any one of clauses 1-9, or the salt thereof, wherein A is:

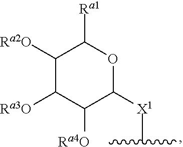

wherein:
R^{a1} is —CH₃, —CH₂OH, or hydrogen;
R^{a2} and R^{a3} are each hydrogen;
R^{a4} is hydrogen, a monosaccharide moiety, or a disaccharide moiety; and
X¹ is O, S, or NH.
Clause 11. The glycolipid of clause 10, or the salt thereof, wherein A is:

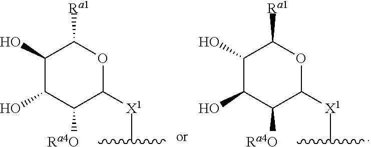

Clause 12. The glycolipid of clause 11, or the salt thereof, wherein A is:

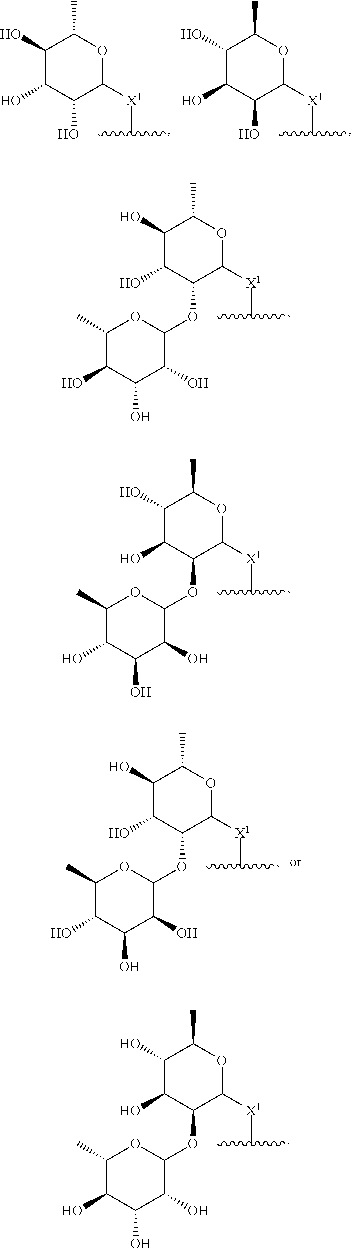

Clause 13. The glycolipid of any one of clauses 10-12, or the salt thereof, wherein X¹ is O.
Clause 14. The glycolipid of any one of clauses 1-13, or the salt thereof, wherein the glycolipid of formula (I) is a glycolipid of formula (I-a), (I-b), (I-aa), (I-bb), (I-ab), or (I-ba):

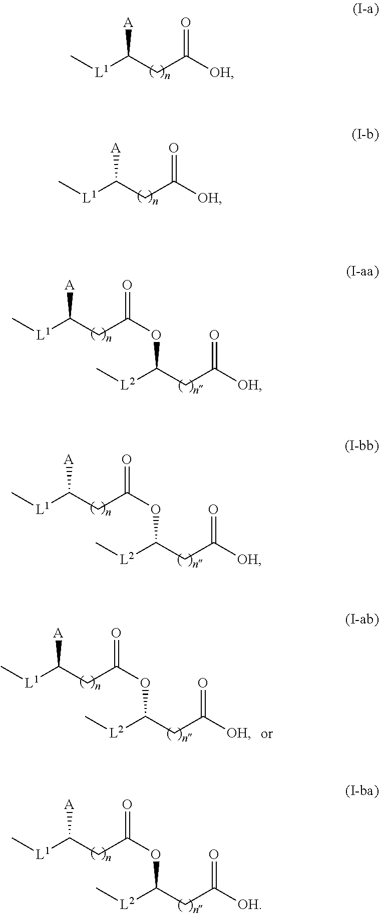

Clause 15. A method of preparing the glycolipid of any one of clauses 1-14, or the salt thereof, wherein n is 0, the method comprising:
   v. preparing an intermediate of formula (I-1B), where X¹ is O, S, or NH:

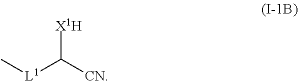

vi. hydrolyzing the intermediate of formula (I-1B) to provide an intermediate of formula (I-1C):

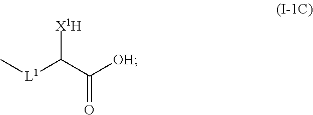

vii. reacting the intermediate of formula (I-1C) with a reagent containing a first hydroxyl protecting group (PG) to provide an intermediate of formula (I-1D):

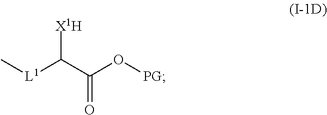

ii. in the presence of a glycosylation promoter, reacting the intermediate of formula (I-1D) with a compound of formula (I-S):

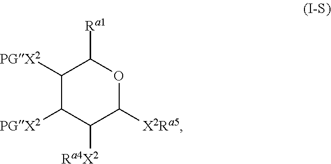

wherein:
   X², at each occurrence, is O or S;
   PG″, at each occurrence is a second hydroxyl protecting group or a thiol protecting group;
   R^{a1} is —CH₃, hydrogen, or —CH₂X²PG″; and
   R^{a4} is PG″, a monosaccharide moiety, or a disaccharide moiety, to provide an intermediate of formula (I-1E):

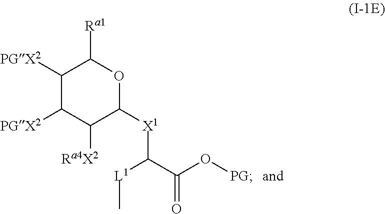

viii. removing the PG and PG″ groups from the intermediate of formula (I-2E) to provide a glycolipid of formula:

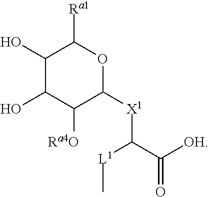

Clause 16. The method of clause 15, where preparing the intermediate of formula (I-1B) comprises reacting a nitrile with an intermediate of formula (I-1A):

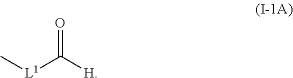

Clause 17. A method of preparing the glycolipid of any one of clauses 1-14, or the salt thereof, wherein n is 2, the method comprising:
vii. preparing an intermediate of formula (I-2B):

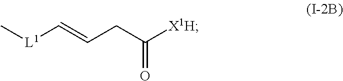

viii. cyclizing the intermediate of formula (I-2B) to provide an intermediate of formula (I-2C:

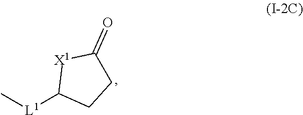

where X¹ is O, S, or NH;
ix. hydrolyzing the intermediate of formula (I-2C) to provide an intermediate of formula (I-2D):

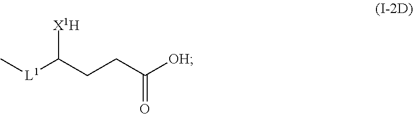

x. reacting the intermediate of formula (I-2D) with a reagent containing a first hydroxyl protecting group (PG) to provide an intermediate of formula (I-2E):

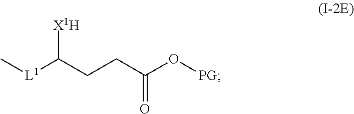

xi. in the presence of a glycosylation promoter, reacting the intermediate of formula (I-2E) with a compound of formula (I-S):

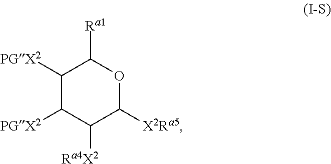

wherein:
   X², at each occurrence is O or S;
   PG″, at each occurrence, is a second hydroxyl protecting group or a thiol protecting group;
   R^{a1} is —CH₃, hydrogen, or —CH₂X²PG″; and
   R^{a4} is PG″, a monosaccharide moiety, or a disaccharide moiety,
to provide an intermediate of formula (I-2F):

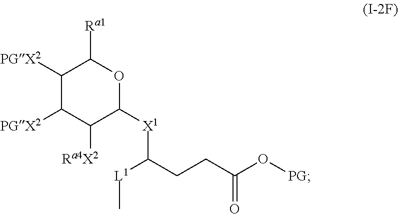

xii. removing the PG and PG″ groups from the intermediate of formula (I-2F) to provide a glycolipid of formula:

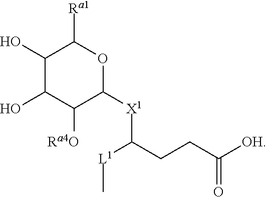

Clause 18. The method of clause 17, where preparing the intermediate of formula (I-2B) comprises reacting a compound of formula (1-2A′) with a compound of formula (I-2A″):

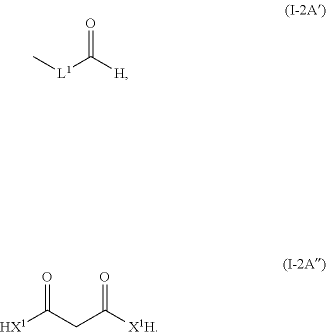

Clause 19. The method of any one of clauses 15-18, wherein the glycosylation promoter comprises Bi(OTf)₃, InBr₃, or BF₃.
Clause 20. The method of any one of clauses 15-19, wherein L¹ is C₂₋₁₈alkylene.

1. A glycolipid of formula (I), or a salt thereof, 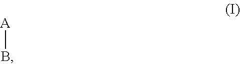
wherein:
A is a monosaccharide, a disaccharide, a trisaccharide, or an amine or thiol derivative thereof;
B is 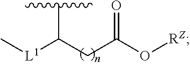
L¹ is C₂₋₁₈alkylene or C₂₋₁₈alkenylene;
n is 0 or 2;
R^{Z} is hydrogen, C₁₋₆alkyl, C₁₋₆haloalkyl, G^{1a}, —C₁₋₆alkylene-G^{1a}, —C₁₋₆alkylene-R^{Y}, or 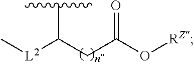
L², at each occurrence, is C₂₋₁₈alkylene or C₂₋₁₈alkenylene;
n″, at each occurrence, is 0 or 2;
R^{Z″}, at each occurrence, is hydrogen, C₁₋₆alkyl, C₁₋₆haloalkyl, G^{1a″}, —C₁₋₆alkylene-G^{1a″}, or —C₁₋₆alkylene-R^{Y″};
R^{Y}, at each occurrence, is —OC₁₋₄alkyl, —OC₁₋₄haloalkyl, —OH, cyano, —SH, —SC₁₋₄alkyl, —SC₁₋₄haloalkyl, —SG^{1a}, —NH₂, —NHC₁₋₄alkyl, —NHG^{1a}, —N(C₁₋₄alkyl)₂, —N(G^{1a})₂, —C(O)OG^{1a}, —C(O)OC₁₋₄alkyl, —C(O)NH₂, —C(O)NHC₁₋₄alkyl, —C(O)N(C₁₋₄alkyl)₂, —C(O)NHG^{1a}, —C(O)N(G^{1a})₂, —SO₂C₁₋₄alkyl, —SO₂G^{1a}, —SO₂NH₂, —SO₂NHC₁₋₄alkyl, or —SO₂N(C₁₋₄alkyl)₂;
R^{Y″}, at each occurrence, is —OC₁₋₄alkyl, —OC₁₋₄haloalkyl, —OH, cyano, —SH, —SC₁₋₄alkyl, —SC₁₋₄haloalkyl, —SG^{1a″}, —NH₂, —NHC₁₋₄alkyl, —NHG^{1a″}, —N(C₁₋₄alkyl)₂, —N(G^{1a″})₂, —C(O)OG^{1a″}, —C(O)OC₁₋₄alkyl, —C(O)NH₂, —C(O)NHC₁₋₄alkyl, —C(O)N(C₁₋₄alkyl)₂, —C(O)NHG^{1a″}, —C(O)N(G^{1a″})₂, —SO₂C₁₋₄alkyl, —SO₂G^{1a″}, —SO₂NH₂, —SO₂NHC₁₋₄alkyl, or —SO₂N(C₁₋₄alkyl)₂; and
G^{1a} and G^{1a″}, at each occurrence, are independently a C₃₋₈cycloalkyl, a 4- to 12-membered heterocyclyl, a 6- to 12-membered aryl, or a 5- to 12-membered heteroaryl, wherein G^{1a} and G^{1a″} are independently optionally substituted with 1-5 substituents independently selected from the group consisting of halogen, oxo, C₁₋₄alkyl, —OC₁₋₄alkyl, —OC₁₋₄haloalkyl, —OH, —SC₁₋₄alkyl, —SC₁₋₄haloalkyl, —SH, —NO₂, —NH₂, —NHC₁₋₄alkyl, —N(C₁₋₄alkyl)₂, cyano, —C(O)OC₁₋₄alkyl, —C(O)NH₂, —C(O)NHC₁₋₄alkyl, —C(O)N(C₁₋₄alkyl)₂, —SO₂C₁₋₄alkyl, —SO₂NH₂, —SO₂NHC₁₋₄alkyl, and —SO₂N(C₁₋₄alkyl)₂.

2. The glycolipid of claim 1, or the salt thereof, wherein the saccharide is a monosaccharide.

3. The glycolipid of claim 2, or the salt thereof, wherein the monosaccharide is allose, altrose, arabinose, fructose, fucose, galactose, glucose, gulose, idose, lxyose, psicose, rhamnose, ribose, 2-deoxy-ribose, ribulose, sorbose, tagatose, talose, xylose, xylulose, or an amine or thiol derivative thereof.

4. The glycolipid of claim 1, or the salt thereof, wherein the saccharide is a disaccharide.

5. The glycolipid of claim 4, or the salt thereof, wherein the disaccharide is cellobiose, chitobiose, dirhamnose, gentiobiose, isomaltose, isomaltulose, lactose, lactulose, laminaribose, leucrose, maltose, maltulose, melibiose, nigerose, sophorose, sucrose, terhalose, turanose, xylobiose, or an amine or thiol derivative thereof.

6. The glycolipid of claim 1, or the salt thereof, wherein the saccharide is a trisaccharide.

7. The glycolipid of claim 6, or the salt thereof, wherein the trisaccharide is cellotriose, isomaltotriose, isopanose, laminaritriose, manninotriose, maltotriose, melezitose, nigerotriose, panose, raffinose, xylotriose, or an amine or thiol derivative thereof.

8. The glycolipid of claim 1, or the salt thereof, wherein the saccharide is a naturally occurring saccharide.

9. The glycolipid of claim 1, or the salt thereof, wherein the saccharide is a synthetically prepared saccharide.

10. The glycolipid of claim 1, or the salt thereof, wherein A is: 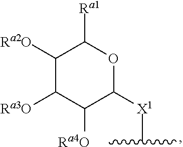
wherein:
R^{a1} is —CH₃, —CH₂OH, or hydrogen;
R^{a2} and R^{a3} are each hydrogen;
R^{a4} is hydrogen, a monosaccharide moiety, or a disaccharide moiety; and
X¹ is O, S, or NH.

11. The glycolipid of claim 10, or the salt thereof, wherein A is:

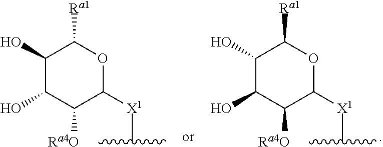

12. The glycolipid of claim 11, or the salt thereof, wherein A is:

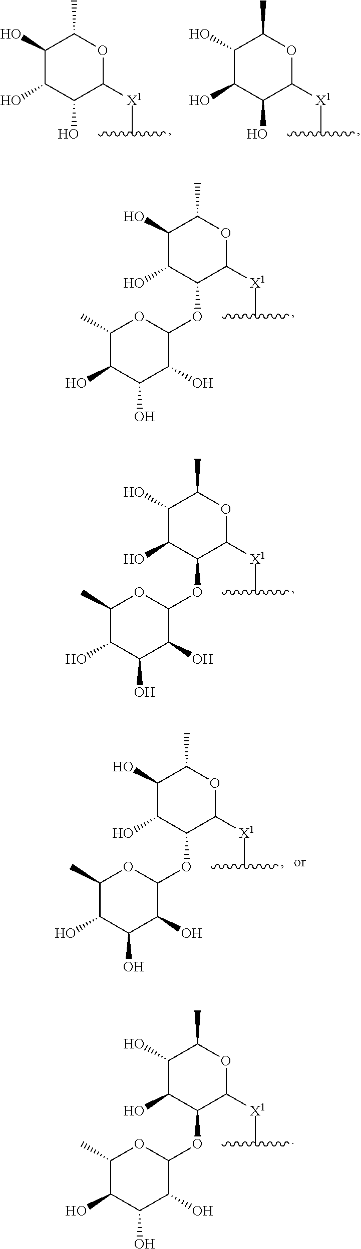

13. The glycolipid of claim 10, or the salt thereof, wherein X¹ is O.

14. The glycolipid of claim 1, or the salt thereof, wherein the glycolipid of formula (I) is a glycolipid of formula (I-a), (I-b), (I-aa), (I-bb), (I-ab), or (I-ba):

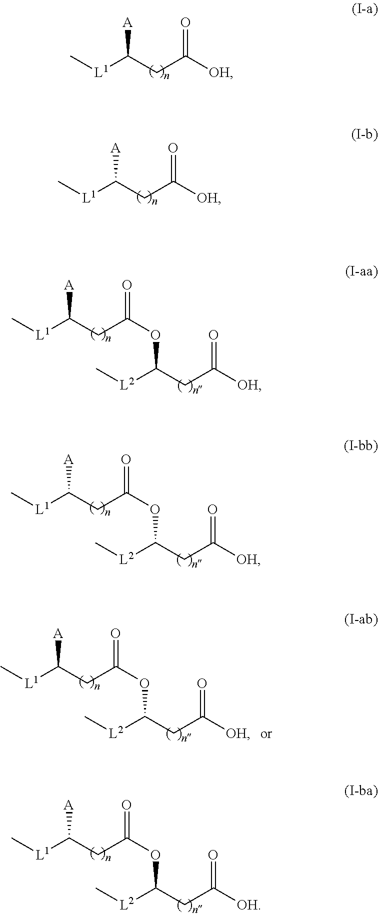

15. A method of preparing the glycolipid of claim 1, or the salt thereof, wherein n is 0,
the method comprising:
i. preparing an intermediate of formula (I-1B), where X¹ is O, S, or NH: 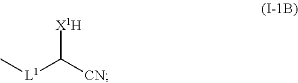
ii. hydrolyzing the intermediate of formula (I-1B) to provide an intermediate of formula (I-1C): 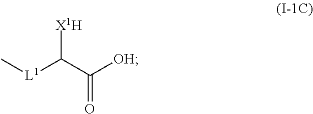
iii. reacting the intermediate of formula (I-1C) with a reagent containing a first hydroxyl protecting group (PG) to provide an intermediate of formula (I-1D): 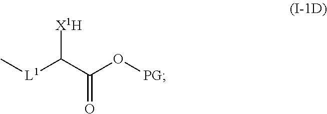
i. in the presence of a glycosylation promoter, reacting the intermediate of formula (I-1D) with a compound of formula (I-S): 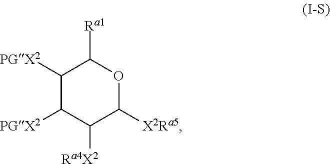
wherein:
X², at each occurrence, is O or S;
PG″, at each occurrence is a second hydroxyl protecting group or a thiol protecting group;
R^{a1} is —CH₃, hydrogen, or —CH₂X²PG″; and
R^{a4} is PG″, a monosaccharide moiety, or a disaccharide moiety,
to provide an intermediate of formula (I-1E): 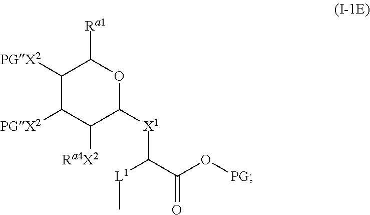
and
iv. removing the PG and PG″ groups from the intermediate of formula (I-2E) to provide a glycolipid of formula:

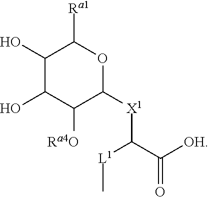

16. The method of claim 15, where preparing the intermediate of formula (I-1B) comprises reacting a nitrile with an intermediate of formula (I-1A):

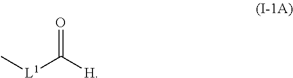

17. A method of preparing the glycolipid of claim 1, or the salt thereof, wherein n is 2,
the method comprising:
i. preparing an intermediate of formula (I-2B): 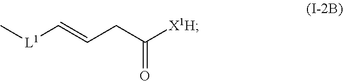
ii. cyclizing the intermediate of formula (I-2B) to provide an intermediate of formula (I-2C: 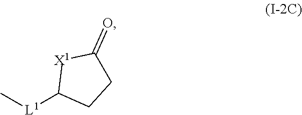
where X¹ is O, S, or NH;
iii. hydrolyzing the intermediate of formula (I-2C) to provide an intermediate of formula (I-2D): 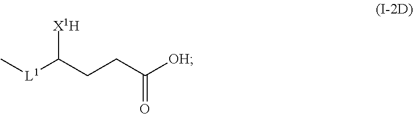
iv. reacting the intermediate of formula (I-2D) with a reagent containing a first hydroxyl protecting group (PG) to provide an intermediate of formula (I-2E): 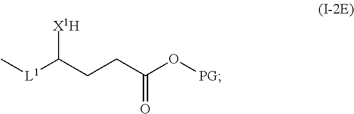
v. in the presence of a glycosylation promoter, reacting the intermediate of formula (I-2E) with a compound of formula (I-S): 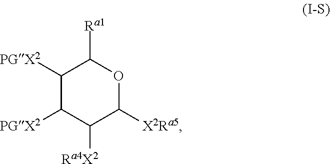
wherein:
X², at each occurrence is O or S;
PG″, at each occurrence, is a second hydroxyl protecting group or a thiol protecting group;
R^{a1} is —CH₃, hydrogen, or —CH₂X²PG″; and
R^{a4} is PG″, a monosaccharide moiety, or a disaccharide moiety,
to provide an intermediate of formula (I-2F): 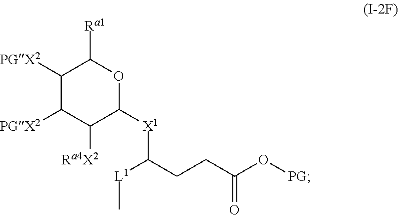
and
vi. removing the PG and PG″ groups from the intermediate of formula (I-2F) to provide a glycolipid of formula:

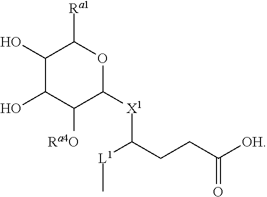

18. The method of claim 17, where preparing the intermediate of formula (I-2B) comprises reacting a compound of formula (I-2A′) with a compound of formula (I-2A″):

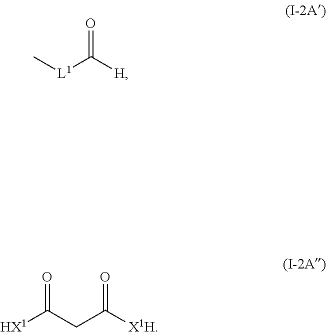

19. The method of claim 15, wherein the glycosylation promoter comprises Bi(OTf)₃, InBr₃, or BF₃.

20. The method of claim 15, wherein L¹ is C₂₋₁₈alkylene.
